(19) United States
(12) Patent Application Publication
(10) Pub. No.: US 2023/0102836 A1
(43) Pub. Date: Mar. 30, 2023

(54) Amoebicidal Compositions for Contact Lens Solutions

(71) Applicant: University of the West of Scotland, Paisley (GB)

(72) Inventors: Fiona Henriquez, Glasgow (GB); Roderick Williams, Glasgow (GB); Ronnie Mooney, Glasgow (GB)

(21) Appl. No.: 17/671,465

(22) Filed: Feb. 14, 2022

Related U.S. Application Data
(63) Continuation of application No. PCT/GB2020/051947, filed on Aug. 14, 2020.

(30) Foreign Application Priority Data
Aug. 15, 2019 (GB) 1911694.6

(51) Int. Cl. A61K 31/661; A61P 33/04

(52) U.S. Cl. CPC A61K 31/661; A61P 33/04

(57) ABSTRACT

Described herein are pharmaceutical compositions and contact lens solutions comprising an Acanthamoeba encystation inhibitor such as a cationic quaternary ammonium compound and a physiologically or pharmaceutically acceptable carrier or excipient, optionally in combination with an Acanthamoeba cytotoxic agent such as an alkylphosphocholine. Such compositions are suitable for use in the treatment of Acanthamoeba infection or the prevention or treatment of Acanthamoeba keratitis.

### CROSS-REFERENCE TO RELATED APPLICATIONS

This application is a continuation of International Application No. PCT/GB2020/051947, filed 14 Aug. 2020, which in turn claims priority to Great Britain Patent Application No. 1911694.6, filed 15 Aug. 2019, the contents of which are hereby incorporated by reference in their entirety.

### TECHNICAL FIELD

This invention relates to a composition, and in particular to a composition for the treatment of Acanthamoeba infection. More particularly, it relates to compositions for use in the treatment of such infection. Even more particularly, it relates to novel compositions of contact lens solutions.

Background Many different species of the opportunistic parasite Acanthamoeba spp. exist and are commonly found in lakes, swimming pools, tap water, and heating and air conditioning units. Acanthamoeba organisms do not generally cause harm to humans but predominantly the T4 genotype, including A. culbertsoni, A. polyphaga, A. castellanii, A. astronyxis, A. hatchetti, A. rhysodes, A. divionensis, A. lugdunensis, and A. lenticulate, are known to cause a serious eye disease if they infect the cornea. Acanthamoeba castellanii is one causative agent of the sight threatening infection Acanthamoeba keratitis (AK), an infection commonly associated with contact lens use and of increasing importance given a recent rise of recorded incidence rates.

Acanthamoeba are free-living protists that exist as either non-replicative cysts protected with a double cellulose cell wall or as replicative trophozoites, which are opportunistic parasites of the cornea, skin, and brain of humans causing diseases called Acanthamoeba keratitis (AK), cutaneous acanthamoebiasis (CA) and granulomatous amoebic encephalitis (GAE) respectively. GAE is extremely rare and fatal, while the more prevalent AK is non-lethal but can lead to sight loss in the infected eye and is a source of emotional and psychological trauma for those infected. Interconversion between trophozoites and cysts are triggered by environmental and chemical stresses (Hay J et al (1994) Eye; 8: 555-563; Roberts C W, Henriquez F L (2010) Exp. Parasitol; 126(1): 91-96) with cyst to trophozoite transformation being responsible for AK resurgences and relapses (Mazur T et al (1995) Trop. Med Parasitol.; 46: 106-108).

Inadequate contact lens management strategies are major contributors to recent increases in AK incidence (Joslin C E et al (2007) Am. J. Ophthalmol.; 144(2): 169-180; Verani J R et al (2009) Emerg. Infect. Dis.; 15(8): 1236-1242), being 15 times higher in the UK than the USA (Stehr-Green J K et al (1989) Am. J. Ophthalmol.; 107: 331-336; Radford C F et al (2002) Br. J. Ophthalmol.; 8_6:536-542) and eight times more prevalent in Scotland than England (Seal D V et al (1999) Cont. Lens Anterior Eye; 22: 49-57). These spatial differences are linked with mode of supply, quality and water usage (Radford et al supra), and the risk is increased as a result of rinsing contact lenses with domestic tap water, recommended to protect the eye from damage by antimicrobials post contact lens cleansing (Kilvington S et al (2004) Investig. Ophthalmol. Vis. Sci.; 45:165-169). Rinsing has not helped maintain eye health, but exposed compliant lens wearers to Acanthamoeba. The lack of anti-Acanthamoeba agents in contact lens cleaning solutions and the anti-microbials being encystation inducers are additional risk factors. Thus, reduction of AK incidence requires a better contact lens cleansing strategy and cleansing formulation.

Currently available treatments of Acanthamoeba infection and AK incidence are harsh and prolonged, not least because of the ability of Acanthamoeba to adapt rapidly to harsh conditions and switch to a resistant cyst form, as well as the lack of available methods for the targeted killing of trophozoites and cysts. In addition, cysts (the source of infection recurrence) can remain viable for years while maintaining their pathogenicity (Mazur T et al (1995) Trop. Med. Parasitul.; 46: 106-108).

Identification of compounds to address this issue remains challenging. Combination therapies are highly recommended for curative treatment of AK with polyhexamethylene biguanide (PHMB), chlorhexidine, propamidine isethionate, dibromopropamidine and hexamidine being commonly used. However, these have proved limited as they fail to prevent encystation (Lorenzo-Morales J et al (2015) Parasite; 22: 10).

Thus, there remains a need for a need for a suitable composition to protect contact lens users from infection by Acanthamoeba, such as A. castellanii, and the development of diseases, such as Acanthamoeba keratitis (AK), that occur as a result of such infection. Accordingly, it is an object of this invention to provide a pharmaceutical composition for use in the treatment of Acanthamoeba infection and the prevention and treatment of Acanthamoeba keratitis (AK) that targets both the trophozoite and cyst forms of Acanthamoeba.

### SUMMARY

In accordance with a first aspect of the invention, there is provided a pharmaceutical composition comprising at least one anti-Acanthamoeba agent and a physiologically or pharmaceutically acceptable carrier or excipient, for use in the treatment of Acanthamoeba infection, wherein the anti-Acanthamoeba agent has a structure according to formula (I)

R¹—[Lₘ]—N⁺(R²)(R³)(R⁴) (I)

wherein

R¹ is an optionally substituted saturated or unsaturated C₁₀-C₂₄ chain;

L is a linker moiety, wherein m=0 or 1; and

R², R³ and R⁴ are independently C₁-C₄ alkyl substituents (preferably n-alkyl), aryl substituents, or, together with an alkylene moiety of an optional linker L, forms a heterocyclic moiety, whilst optionally one of R², R³ and R⁴ may be a straight-chain, unsubstituted aliphatic hydrocarbon chain of up to 22 having a definition corresponding to that of R¹.

N⁺ as is the convention represents an ammonium ion which typically has a counterion by way of an ammonium salt, such as chloride, bromide or iodide or any other suitable counterion, or may have a counterion provided in an L-group forming a zwitterionic species.

The Agent of the Acanthamoeba infection may be A. castellanii.

In an associated aspect of the invention, there is provided a pharmaceutical composition comprising a cationic quaternary ammonium compound (QAC) and a physiologically or pharmaceutically acceptable carrier or excipient, for use in the treatment of Acanthamoeba infection.

Expressed in another way, the invention resides in use of a pharmaceutical composition in the manufacture of a medicament for the treatment of Acanthamoeba infection, the pharmaceutical composition comprising an anti-Acanthamoeba agent as defined above and a physiologically or pharmaceutically acceptable carrier or excipient. The agent of the Acanthamoeba infection may be A. castellanii.

Expressed in yet another way, the invention encompasses a method for the treatment of Acanthamoeba infection comprising the administration of a therapeutically effective amount of a pharmaceutical composition, the pharmaceutical composition comprising at least one anti-Acanthamoeba agent as defined above and a physiologically or pharmaceutically acceptable carrier or excipient. The agent of the Acanthamoeba infection may be A. castellanii.

In a second aspect of the invention, there is provided a contact lens solution comprising at least anti-Acanthamoeba agent, as described above, and an acceptable carrier or excipient.

In a third aspect of the invention, there is provided a pharmaceutical composition comprising: a) an Acanthamoeba encystation inhibitor; b) an Acanthamoeba cytotoxic agent; and c) a physiologically or pharmaceutically acceptable carrier or excipient.

Expressed in another way, this aspect encompasses use of the pharmaceutical composition described hereinabove in the manufacture of a medicament for the treatment of A. castellanii infection or the prevention or treatment of Acanthamoeba keratitis.

Expressed in a yet further way, this aspect also encompasses a method for the treatment of A. castellanii infection or the prevention or treatment of Acanthamoeba keratitis comprising the administration of a therapeutically effective amount of the pharmaceutical composition described herein above.

In a fourth aspect of the invention, there is provided a contact lens solution comprising: a) an Acanthamoeba encystation inhibitor; b) an Acanthamoeba cytotoxic agent; and c) an acceptable carrier or excipient as described hereinabove. In one example, the Acanthamoeba encystation inhibitor and/or the Acanthamoeba cutotoxic agent are selective for A. castellanii.

### BRIEF DESCRIPTION OF THE DRAWINGS

FIGS. 1A-1C show the extrusion of cytoplasmic constituents of A. castellanii trophozoites to the external milieu after QAC treatment. [K⁺] (FIG. 1A). DNA (FIG. 1B) and Protein (FIG. 1C) released from 10⁵ A. castellanii trophozoites after treatment without (control) and with QAC18 at 37.5 μg/ml for 48 h into spent medium and measured with the Atomic Absorption Spectroscopy (view A), the Nanodrop® at 260 nm (view B) and at 280 nm (view C) respectively were increased 3.6-fold, 2.6-fold and 3.6-fold respectively. Data is mean±SD; n=4 independent experiments performed in triplicates. Student's t-test showed significant difference between mean [K⁺], [DNA] and [Proteins] at baseline (control and medium) and QAC18 at p<0.01.

FIGS. 2A-2C show that QAC18 induces a cyst-like morphology on A. castellanii. Acanthamoeba trophozoites (10 cells/ml) incubated without (FIG. 2A), with QAC18 at 37.5 μg/ml for 1 h at 25° C. (FIG. 2B), and 0.5% SDS for 30 min (FIG. 2C). QAC18 reduced trophozoites sizes from 29.02±0.23 (FIG. 2A) to 7.87 f 0.93 (FIG. 2B) which disintegrated after 30 min treatment with SDS (FIG. 2C).

FIGS. 3A and 3B show the QAC-DNA interaction. The absorbance of genomic DNA (0.6 ng/ml) extracted from A. castellani trophozoites measured at 260 nm with the Nanodrop after incubating for 15 minutes with 0 ng/ml, 0.6 ng/ml, 6 ng/ml or 12 ng/ml of QAC12 (FIG. 3A) or QAC16 (FIG. 3B) to give ratios of 1:0, 1:1, 1:10 and 1:20. DNA absorbance increased and decreased with increased [QAC18] and [QAC12] respectively. Data is mean absorbance at 260 nm±SD; n=4 independent experiments performed in triplicates. Student's t-test showed significant difference between mean absorbance at 1.1, 1:10 and 1:20 relative to 1:0 at p<0.01 for QAC18 (FIG. 3A) and QAC12 (FIG. 3B) respectively.

FIG. 4 shows that QAC12 increased A. castellanii cell density in vitro. Acanthamoeba trophozoites (10⁵ cells/ml. n=5/treatment) treated with 37.50 μg/ml QAC12 doubly serially diluted to 0.07 μg/ml for 96 h at 25° C. Cell viability estimated by the alamarBlue® assay showed dose-dependent increase in mean absorbance, a measure for cell density, which peaked at 37.7 μg/ml to produce a 4-fold increase absorbance at 570 nm relative to control cells without QAC12 added (black bar). Data is mean absorbance at 570 nm±SD; n=4 independent experiments performed in triplicates. Student's t-test showed significant difference between mean absorbance at baseline (black bar) and QAC12 concentration >4.86 μg/ml at p<0.01.

FIG. 5 shows that QAC12 delayed A. castellanii trophozoite-cyst conversion. Acanthamoeba trophozoites (10⁵ cells/ml) incubated for up to 192 h in encystment medium inoculated with (dark grey) and without (light grey) QAC12 at 25° C. The number of trophozoites remaining estimated microscopically and expressed as a percent of total trophozoites at the start of the assay. Data is mean absorbance at 570 nm±SD; n=4 independent experiments performed in triplicates. Student's t-test showed significant difference between % trophozoites with (grey bars) and without (light grey bars) QAC12 at p<0.01.

FIGS. 6A-6D show that QAC12 increased APC16 efficacy but not QAC18 efficacy in vitro. Acanthamoeba trophozoites (10⁵ cells/ml. n=5/treatment) treated with 100 μg/ml doubly diluted to 0.06 μg/ml of QAC18 (a,b) or APC16 (c,d) for 96 h at 25° C. Cell viability estimated by the alamarBlue assay showed dose-dependent decreases in mean absorbance, a measure for cell density, for (FIG. 6A) QAC18 alone at the same concentration (closed circles, FIG. 6A, FIG. 6B) or in combination (FIG. 6A) QAC12 at 18.75 μg/ml (open circle, FIG. 6A) or (FIG. 6B) QAC12 at 37.35 μg/ml (open circle, FIG. 6B). Similarly. APC16 alone at the same concentration (closed circle, FIG. 6C, FIG. 6D) in combination with QAC12 at 18.75 μg/ml (open squares, d) QAC12 at 37.35 μg/ml (open squares, FIG. 6C). Data are mean absorbance at 595 nm t SD; n=4 independent experiments performed in triplicates. IC50s were not statistically significant between QAC12 at both concentrations and QAC16 (p>0.05, FIG. 6A, FIG. 6B) but significant with APC16 (p<0.05, FIG. 6C, FIG. 6D).

FIGS. 7A-7D show that QAC12, not the active analogues, was synergistic with APCs in vitro. Acanthamoeba trophozoites (10⁵ cells/ml. n=5/treatment) treated with 100 μg/ml doubly diluted to 0.06 μg/ml of QAC12 (FIG. 7A), QAC12 (FIG. 7B), QAC16 (FIG. 7C) and QAC18 (FIG. 7D) with APC 12 (i), APCI 4 (ii) and APC16 (iii) for 96 h at 25° C. Cell viability estimated by the alamarBlue assay the dose-response curves used to produce a surface analysis (right panel) and the interaction profile determined using the Bliss model. The degree of interaction characterised by the QCScore heuristic score are produced as heat map (left panel). The colour of each cell in the heatmap and the surface analysis represents the assay response at that dose combination; red to orange, antagonistic: yellow to green, additive and blue, synergistic.

FIGS. 8A-8H show the integrity of A. castellanii cyst and cytotoxicity to QACs. Acanthamoeba trophozoites (10⁵ cells/ml) incubated in encystment medium for 7 days, stained with calcofluor white for 30 minutes observed with the DAPI (FIG. 8A) and DIC (FIG. 8B) filters of the EVOS microscope. Merged image of a single cyst (DIC+calcofluor white stained cells, FIG. 8C) and their respective individual calcofluor white (FIG. 8D) and DIC (FIG. 8E) images. Toxicity of QACs to A. castellanii cyst assessed using the trypan blue assay with viable (FIG. 8F) and dead (FIG. 8G) cysts without and with respectively, cytosolic blue staining. IC₅₀ of cysts treated with QAC16 and QAC18 ranging from 37.5 μg/ml to 0.07 μg/ml for 24, 48 and 72 h respectively (FIG. 8H). Data in (FIG. 8H) is mean±SD; n=4 independent experiments performed in triplicates. Student's t-test showed significant difference between 48 h and 72 h for QAC18 relative to 24 h at p<0.01. No significant difference was observed for QAC16 at the same duration ad degree of significance.

FIGS. 9A-9B show the extrusion of cytoplasmic constituents of A. castellani cysts to the external milieu after QAC treatment. DNA (FIG. 9A) and Protein (FIG. 9B) released after the incubation of 10⁵ A. castellanii cysts, with (QAC18) and without (control) QAC18 at 37.5 μg/ml for 48 h, measured in spent medium using the Nanodrop® at 260 nm (FIG. 9A) and at 280 nm (FIG. 9B) respectively. Data is mean absorbance at 260 nm (FIG. 9A) and 280 nm (FIG. 9B)±SD respectively: n=4 independent experiments performed in triplicates. Student's t-test showed significant difference between difference between mean [DNA] and [Proteins] at baseline (control and medium) and QAC18 at p<0.01.

### DETAILED DESCRIPTION

The present invention is concerned with according to one aspect a pharmaceutical composition comprising at least one anti-Acanthamoeba agent as defined above and to a use of a an anti-Acanthamoeba agent (or a pharmaceutical composition comprising the anti-Acanthamoeba agent) in the manufacture of a medicament for the treatment of an Acanthamoeba infection. It may further relate to a method for the treatment of Acanthamoeba infection by administration of the anti-Acanthamoeba agent (or a pharmaceutical composition comprising the anti-Acanthamoeba agent) in a therapeutically effective amount thereof.

In another aspect, the invention is concerned with a contact lens solution comprising at least one anti-Acanthamoeba agent along with an acceptable carrier or excipient.

In third and fourth aspects, the invention relates respectively to a pharmaceutical composition and to a contact lens solution in each case comprising a) an Acanthamoeba encystation inhibitor, b) an Acanthamoeba cytotoxic agent and c) an acceptable carrier or excipient. In one particular embodiment, the Acanthamoeba cytotoxic agent is or comprises an anti-Acanthamoeba agent as defined here.

In each case, the composition or solution (or the anti-Acanthamoeba agent) is preferably an acanthamoebocide. Preferably, the anti-Acanthamoeba agent (or composition or solution comprising the anti-Acanthamoeba agent) preferably kills both trophozoite and cyst forms of Acanthamoeba, including A. castellanii.

In a particular embodiment, the pharmaceutical compositions and/or contact lens solutions described herein may be used to prevent or treat Acanthamoeba keratitis.

The Acanthamoeba infections may be caused by any one of a number of Acanthameoba organisms or infective agents, such as A. culbertsoa, A. polyphaga, A. castellani, A. astronyxis, A. hatchetti, A. rhysodes, A. divionensis. A. lugdunensis, and A. lenticulate. In one particular embodiment, the infective agent is A. castellanii.

The invention is especially directed to the prevention or treatment of Acanthamoeba keratitis arising from A. castellanii infection.

The pharmaceutical composition may be formulated in any suitable way according to the particular application and treatment of the Acanthamoeba infection as required. In one particular embodiment, the pharmaceutical composition may be formulated for intraocular application. It will be appreciated that such formulation may be an aqueous or media solution or suspension for application as drops, or a paste or similar for topical application, to the site of infection.

The contact lens solution may be for use in the treatment of Acanthamoeba infection, or prevention or treatment of Acanthamoeba keratitis. The contact lens solutions described herein may be formulated in any suitable way and configured to include an anti-Acanthamoeba agent as defined herein.

Alternatively or in addition, the contact lens solution may be for use in cleaning contact lenses.

As discussed above, the anti-Acanthamoeba agent has a structure according to formula (I)

R¹-[Lₘ]-N⁺(R²)(R³)(R⁴) (I)

wherein

R¹ is an optionally substituted saturated or unsaturated C₁₀-C₂₄ aliphatic hydrocarbon chain;

L is a linker moiety, wherein m=0 or 1; and

R², R³ and R₄ are independently C₁-C₄ alkyl substituents (preferably n-akyl), aryl substituents, or, together with an alkylene moiety of an optional linker L, forms a heterocyclic moiety, whilst optionally one of R², R³ and R⁴ may be a straight-chain, unsubstituted aliphatic hydrocarbon chain of up to 22 having a definition corresponding to that of R¹.

The agent preferably has a counter-ion comprising a halide moiety such as fluoride, chloride, bromide or iodide or is a zwitterionic agent having a counter charge provided by an optional linker, L, moiety.

Preferably R¹ is a straight-chain, unsubstituted aliphatic hydrocarbon chain.

Preferably R¹ comprises an aliphatic chain of up to 22, preferably up to 20 and more preferably up to 18 carbon atoms. More preferably, R¹ comprises a chain of at least 12 carbon atoms.

Most preferably, R¹ is an unsubstituted straight-chain aliphatic hydrocarbon of 12, 14, 16 or 18 carbons. Most preferably, the R¹ group comprises at least 14 carbons and preferably has 14, 16 or 18 carbons.

Optionally, the R¹ group is saturated or unsaturated. In one embodiment, the R¹ group is saturated. In another embodiment, the R¹ group is partially unsaturated, for example having 1 to 4 double bonds, preferably provided in the terminal portion (e.g. the free end or free half) of the aliphatic hydrocarbon chain, more preferably among the terminal 6 carbons of the free end of the aliphatic hydrocarbon chain. More preferably, the R¹ group has 1 or 2 double bonds among the terminal 6 carbons of the free end of the aliphatic hydrocarbon chain, more preferably among the terminal 4 carbon atoms and preferably between the terminal two carbon atoms. Preferably, just one double bond is provided which is preferably between the terminal two carbon atoms. It is believed that some unsaturation in the terminal portion of the R¹ group enhances the efficacy of the anti-Acanthamoeba agent against Acanthamoeba infection.

Preferably, for any of the embodiments, the R², R³ and R⁴ groups are each not an alkylbenzyl moiety such as ethylbenzyl, preferably none of the R², R³ and R; groups comprise an alkylbenzyl moiety and preferably are each absent any aryl groups.

The R², R³ and R⁴ groups are preferably methyl groups.

In a first general embodiment, m=0 and there is no linker moiety between the R¹— group and the —N⁺(R²)(R³)(R⁴) head group (the terms —N⁺(R²)(R³)(R⁴), NR₃ and ‘head group’ being used interchangeably hereafter). The anti-Acanthameoba agent according to this first general embodiment may be termed QACs.

In a second general embodiment, m=1 and the linker moiety may comprise an aliphatic heterocycle (e.g. pyrrolidine, piperidine) directly or indirectly (e.g. by an aliphatic straight chain hydrocarbon moiety of up to 3 carbons, e.g. by a methylene, ethylene or propylene group) linked to the —N⁺(R²)(R³)(R⁴) or incorporating the N and R² of the head group, or may be an aromatic heterocycle (e.g. azole, diazole, triazole, pyridine, pyrimidine) or an aromatic hydrocarbon (e.g. benzene, pyran) indirectly (e.g. by an aliphatic straight chain hydrocarbon moiety of up to 3 carbons, e.g. by a methylene, ethylene or propylene group) linked to the —N⁺(R²)(R³)(R⁴).

In a third general embodiment, m=1 and the linker comprises an alkylphosphate group so as to form a —O—P(O₂)⁻O—R⁵— linker between the R¹ group and the head group, where R⁵ may comprise any suitable alkylene group such as a substituted or unsubstituted, straight-chain or branched —(CH₂)ₚ— group, where p=from 1 to 6, preferably 2. Optionally, the —(CH₂)ₚ— group is a branched alkyl group which forms an aliphatic heterocycle with the N of the head group. Preferably, the —(CH₂)ₚ— group is an unsubstituted straight chain alkylene group of 1 to 6 carbons, e.g. 2, 3 or 4 carbon atoms, preferably 2 carbon atoms. The compounds of this general embodiment may be referred to hereafter as APC agents, which is meant to include alkylphosphocholines (APCs) [having a linker group —O—P(O₂)O—R⁵— where R⁵ is an ethylene group] and APC analogues [where R⁵ is other than an ethylene group, such as a C₃-C₆ alkylene moiety].

The anti-Acanthameoba agent is preferably of the first or third general embodiments.

In a preferred embodiment of the first general embodiment, a QAC anti-Acanthameoba agent, the R¹ group is a C₁₄, C₁₆ or C₁₈ straight-chain, unsubstituted aliphatic hydrocarbon chain. Optionally, the R¹ group has a degree of unsaturation as defined above, e.g. a double bond between the terminal two carbons of the free end of the group, but is preferably a saturated alkyl group. In one embodiment, the QAC anti-Acanthameoba agent is a dimer having an R² group corresponding to the R¹ group. Preferably, however, R², R³ and R⁴ are independently C₁-C₄ alkyl substituents and are each preferably methyl groups. Preferably, a QAC anti-Acanthameoba agent is formulated as an ammonium bromide.

Particularly preferred QAC anti-Acanthameoba agents include: tetradecyltrimethyl ammonium bromide; hexadecyltrimethyl ammonium bromide; and octadecyltrimethyl ammonium bromide.

In a preferred embodiment of the invention, there is provided a pharmaceutical composition for the prevention or treatment of Acanthamoeba, infection derived from A. castellanii by administering an effective dose to a patient in need thereof, the composition comprising a preferred QAC anti-Acanthameoba agent as defined above. It has surprisingly been found that certain preferred QAC anti-Acanthamoeba agents are effective at killing both trophozoite and cyst forms of A. castellani.

Further, in another preferred embodiment, there is provided a use of a preferred QAC anti-Acanthamoeba agent as defined above for inhibiting or preventing ocular Acanthamoeba infection by A. castellanii by providing the QAC anti-Acanthamoeba agent as a component of a contact lens solution. There is further provided a use of said contact lens solution for inhibiting or preventing ocular Acanthamoeba infection by of A. castellanii.

In a preferred embodiment of the third general embodiment, an APC agent, the R¹ group is a C₁₂, C₁₄, C₁₆ or C₁₈ straight-chain, unsubstituted aliphatic hydrocarbon chain. Optionally, the R¹ group is a saturated alkyl group or, alternatively has a degree of unsaturation as defined above, e.g. a double bond between the terminal two carbons of the free end of the group. Preferably, the R⁵ group is an ethylene moiety.

Particularly preferred APC agents include: tetradecylphosphocholine; hexadecylphosphocholine; and octadecylphosphocholine.

In third and fourth aspects of the invention, as discussed above, are a pharmaceutical composition and a contact lens solution in each case comprising a) an Acanthamoeba encystation inhibitor, b) an Acanthamoeba cytotoxic agent and c) an acceptable carrier or excipient.

The Acanthamoeba cytotoxic agent may be any agent capable of killing Acanthamoeba, especially A. castellani, in its trophozoite form. The Acanthamoeba cytotoxic agent may be, for example, one or a combination of a pentamidine isethionate (PMD), polyhexamethylene biguanide (PHMB), chlorhexidine, brolene, hexamidine and preferably an anti-Acanthamoeba agent as defined by Formula I above. Preferably, the Acanthamoeba cytotoxic agent comprises or more preferably is one or a combination of an anti-Acanthamoeba agents as defined by Formula I above, more preferably a QAC anti-Acanthamoeba agent or an APC agent according to the first and third general embodiments above, still more preferably an APC agent and most preferably an APC agent, the R¹ group is a C₁₂, C₁₄, C₁₆ or C₁₈ straight-chain, unsubstituted aliphatic hydrocarbon chain and R⁵ is an ethylene group. Examples of a suitable Acanthamoeba cytotoxic agent include tetradecylphosphocholine and hexadecylphosphocholine.

The Acanthamoeba encystation inhibitor may be any compound suitable for inclusion as a pharmaceutical composition or a contact lens solution which is capable of inhibiting the encystation of the Acanthamoeba, such as A. castellanii from its trophozoite form to its cyst form. Preferably, the encystation inhibitor is an encystation delay or prevention agent which preferably serves to delay or prevent encystation, thereby maintaining (e.g. for a longer period) the Acanthamoeba in its trophozoite form. While it is an option, preferably, the encystation inhibitor does not elicit its effect by blocking the pathway (or metabolic pathway) to encystation (e.g. by binding to or blocking enzymes essential for encystation such as cellulose synthesis enzymes including glycogen phosphorylase. UDP-glucose pyrophosphorylase and cellulose synthase or by blocking or modulating autophagy or autolysosome formation, e.g. through antagonism of enzymes essential to those processes, such as phophoinositide 3-kinase or MAP kinase), but preferably instead provides favourable conditions for trophozoite maintenance, such as by providing or acting as an energy substrate for Acanthamoeba. In a particularly preferred embodiment, the encystation inhibitor is other than 3-methyladenine. LY294002, Wortmannin, Balfilomycin A1 or Chloroquine. Thus, preferably, the encystation inhibitor is an ensystation delay or prevention agent and is not an encystatation pathway blocker (e.g. a non-encystation pathway blocker). Preferably, the Acanthamoeba encystation inhibitor is not highly cytotoxic to Acanthomoeba organisms or is provided in a dose or concentration that is not cytotoxic to the Acanthamoeba organism but of sufficient dose or concentration to inhibit encystation. Preferably, the Acanthamoeba encystation inhibitor is an energy substrate for an Acanthamoeba organism. Preferably, the Acanthamoeba encystation inhibitor has a structural feature that mimics or resembles a structural feature of the Acanthamoeba cytotoxic agent. For example, in the case of an Acanthamoeba cytotoxic agent having a structure as defined by Formula I, the Acanthamoeba encystation inhibitor may comprise a straight chain aliphatic hydrocarbon moiety.

In a preferred embodiment, especially in relation to the preferred APC agent forms of the Acanthamoeba cytotoxic agent referred to above, the Acanthamoeba encystation inhibitor has a structure define by Formula II:

R⁶-[L_{q}]-N⁺(R₇)(R⁸)(R⁹) (II)

wherein

R¹ is an optionally substituted saturated or unsaturated C₆-C₁₄ aliphatic hydrocarbon chain;

L is a linker moiety, wherein q=0 or 1; and

R⁷, R⁸ and R⁹ are independently C₁-C₆ alkyl substituents, aryl substituents, or, together with an alkylene moiety of an optional linker L, forms a heterocyclic moiety, whilst optionally one of R⁷, R⁸ and R⁹ may be a straight-chain or branched-chain, unsubstituted aliphatic hydrocarbon chain having a definition corresponding to that of R⁶.

The Acanthamoeba cytotoxic agent preferably has a counter-ion comprising a halide moiety such as fluoride, chloride, bromide or iodide or is a zwitterionic agent having a counter charge provided by an optional linker, L, moiety.

In one option, q=1 and the linker moiety may comprise an aliphatic heterocycle (e.g. pyrrolidine, piperidine) directly or indirectly (e.g. by an aliphatic straight chain hydrocarbon moiety of up to 6 carbons, e.g. by a methylene, ethylene or propylene group) linked to the —N⁺(R⁷)(R⁸)(R⁹) or incorporating the N and R⁷ of the head group, or may be an aromatic heterocycle (e.g. azole, diazole, triazole, pyridine, pyrimidine) or an aromatic hydrocarbon (e.g. benzene, pyran) indirectly (e.g. by an aliphatic straight chain hydrocarbon moiety of up to 6 carbons, e.g. by a methylene, ethylene or propylene group) linked to the —N⁺(R⁷)(R⁸)(R⁹).

In a second option, q=1 and the linker comprises an alkylphosphate group so as to form a —O—P(O₂)—O—R¹⁰— linker between the R⁶ group and the head group, where R¹⁰ may comprise any suitable alkylene group such as a substituted or unsubstituted, straight-chain or branched —(CH₂)ᵣ— group, where r=from 1 to 6, for example 2 or 6. Optionally, the —(CH₂)ᵣ— group is a branched alkyl group which forms an aliphatic heterocycle with the N of the head group. The Acanthamoeba cytotoxic agent according to this option may include alkylphosphocholines (APCs) [having a linker group —O—P(O₂)O—R¹⁰— where R¹⁰ is an ethylene group] and APC analogues [where R¹⁰ is other than an ethylene group, such as a C₃-C₆ alkylene moiety].

In one embodiment of the second option above, the R⁶ group is a C₆ to C₁₄ branched aliphatic hydrocarbon chain, preferably fully saturated. Optionally, the R¹⁰ group of the linker is a C₃-C₆ alkylene moiety. Optionally, the R⁸, R⁸ and R⁹ are independently C₃-C₆ alkyl substituents having sec or tert structure, such as isopropyl, s-butyl or t-butyl.

In a further, third, preferred, option, q=0 and there is no linker moiety between the R⁶— group and the —N⁺(R⁷)(R⁸)(R⁹) head group (the terms —N⁺(R⁷)(R⁸)(R⁹), NR₃ and ‘head group’ being used interchangeably hereafter). The Acanthamoeba cytotoxic agent according to this option may be termed QACs. Preferably, according to this preferred embodiment, the R⁶ group is a straight-chain, saturated, unsubstituted aliphatic hydrocarbon chain having 6 to 12 carbons, preferably, 8, 10 or 12, more preferably 10 or 12 and most preferably 12. Preferably, for any of the embodiments of the encystation inhibitor of Formula II, the R⁷, R⁸ and R⁹ groups are each not an alkylbenzyl moiety such as ethylbenzyl, preferably none of the R⁷, R⁸ and R⁹ groups comprise an alkylbenzyl moiety and preferably are each absent any aryl groups. Preferably the R¹, R⁸ and R⁹ groups are methyl groups. Preferably, the Acanthamoeba encystation inhibitor is a dodecyltrimethylammonium salt, such as the bromide salt thereof.

According to a particularly preferred embodiment of the present aspects of the invention, a pharmaceutical composition and a contact lens solution in each case comprises an Acanthamoeba encystation inhibitor selected from a QAC Acanthamoeba cytotoxic agent according to Formula II in which the R⁶ group is a straight-chain, saturated, unsubstituted aliphatic hydrocarbon chain 8, 10 or 12 carbons and an Acanthamoeba cytotoxic agent selected from an APC agent (as defined above) according to Formula 1, in which the R¹ group is a C₁₂, C₁₄, C₁₆ or C₁₈ straight-chain, unsubstituted aliphatic hydrocarbon chain and R⁵ is an ethylene group. In one particular embodiment, the composition or solution comprises a dodecyltrimethylammonium salt as the Acanthamoeba encystation inhibitor and a hexadecylphophocholine or octadecylphosphocholine as the Acanthamoeba cytotoxic agent.

The combination of Acanthamoeba encystation inhibitor and Acanthamoeba cytotoxic agent are believed to behave synergistically to enhance the efficacy of the combination against Acanthamoeba infection. It is believed that the Acanthamoeba encystation inhibitor may act as an energy substrate for the Acanthamoeba organism thereby maintaining the trophozoite form for an extended period allowing the extended action of the Acanthamoeba cytotoxic agent and that there is further synergy demonstrated by the relative structural similarity, which is believed may enhance the trophozoite maintenance period.

In a further, related, aspect, there is provided use of an Acanthamoeba encystation inhibitor as defined above to inhibit encystation of an Acanthamoeba organism. In a further related aspect, there is provided a method of treating or preventing Acanthamoeba infection, the method comprising administering to a patient in need thereof, an effective amount of a pharmaceutical composition as defined above.

According to the method of treatment or pharmaceutical composition, or indeed the contact lens solution according to these third, fourth and subsequent aspects of the invention, the Acanthamoeba encystation inhibitor and Acanthamoeba cytotoxic agent may be administered or formulated together, or separately. If administered together, the Acanthamoeba cytotoxic agent may optionally be formulated in a delayed and/or sustained release formulation whilst the Acanthamoeba encystation inhibitor may optionally be formulated in a sustained release formulation. If administered separately, it is preferred that they are administered such that the Acanthamoeba encystation inhibitor is administered before the Acanthamoeba cytotoxic agent, e.g. immediately before, preferably up to 12 hours before, such as no more than 6 hours and more preferably up to 1 hour before. Optionally, there is a delay of 30 minutes prior to administration of the Acanthamoeba cytotoxic agent.

In one possible treatment regimen, a Acanthamoeba encystation inhibitor, such as a QAC Acanthamoeba encystation inhibitor having a straight-chain C₁₂ aliphatic chain may be administered with a first Acanthamoeba cytotoxic agent, such as an APC having A C₁₆ or C₁₈ aliphatic chain moiety in order to kill an increased proportion of the Acanthamoeba organism in its trophozoite form and then an anti-Acanthamoeba agent effective against the cyst form, such as a QAC anti-Acanthamoeba agent having a C₁₈ aliphatic chain moiety, to kill remaining Acanthamoeba organism in its cyst form.

It will be appreciated that the compositions for the uses described herein may comprise as, or in addition to the active ingredient, an acceptable excipient or diluent, any suitable binder, lubricant, suspending agent, solubilising agent or other materials well known to those skilled in the art. Such materials should be non-toxic and should not interfere with the efficacy of the active ingredient. Acceptable carriers or diluents for therapeutic use are well known in the pharmaceutical art, and are described, for example, in Remington' Pharmaceutical Sciences, Mack Publishing Co. (A. R. Gennaro edit. 1985).

The active ingredient is defined as an anti-Acanthameoba agent, which is preferably a quaternary ammonium compound (QAC) or an alkylphosphocholine (APC) agent as described herein.

The invention will now be described in more detail by way of the following non-limiting examples.

### Examples

Through the use of cytotoxic screening assays to test the anti-Acanthamoeba activities of APC and QAC analogues against Acanthamoeba trophozoites and cysts, individually and in combination, the Applicant has found that APCs and QACs with alkyl carbon chain lengths ranging from 14-18 carbons are effective anti-acanthamoebocides individually, even against A. castellanii cysts, producing death by leakage and DNA compacting. Surprisingly, QAC12 was an energy substrate that increased biomass, delayed encystation and increased the toxicity of APC16 and APC18 against trophozoites.

### Materials and Methods

Cell Lines Used in this Study

Acanthamoeba castellanii ATCC 50370 trophozoites and cysts were cultured in Peptone Glucose (PG) medium and encystment medium (Chagla A H, Griffiths A J (1974) Microbiology: 85(1): 139-145) respectively at 25° C. Cysts were prepared by incubating trophozoites for 8 days in encystment medium and integrity validated with calcofluor (0.25 μg/ml) assay (Gatti S et al (2010) J. Med. Microbiol.; 59: 1324-1330) or sodium dodecyl sulfate (SDS, 0.5% w/v) disintegration assay (Dudley R et al (2005) Acta Trop.; 9: 100-8) and microscopic observation.

Chemicals Used in this Study

QAC and APC analogues used in this study are detained in Table 1.

TABLE 1
Structures APCs and QACs

| | | Molecular | Number | | |
|---|---|---|---|---|---|
| | Chemical | weight | of | Overall | |
| Compound | name | (g/mol) | carbons | Charge | Structure |
| APC8 | Octyl- phospho- choline | 295.4 | 8 | Zwitterionic | 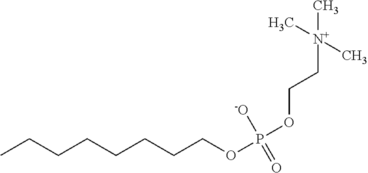 |
| APC10 | Decyl- phospho- choline | 323.4 | 10 | Zwitterionic | 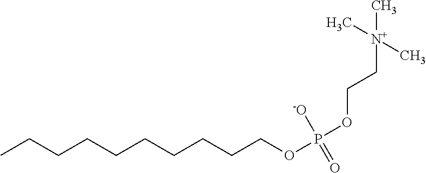 |
| APC12 | Dodecyl- phospho- choline | 351.5 | 12 | Zwitterionic | 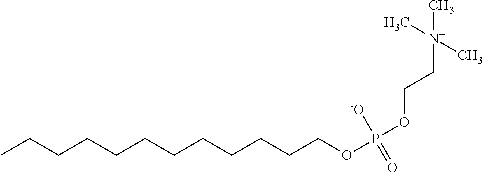 |
| APC12P6C | Dodecyl- phospho- choline | 393.4 | 12 | Zwitterionic | 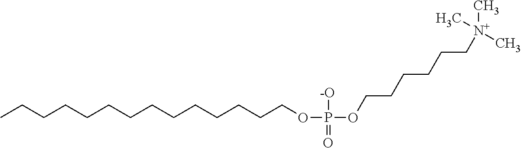 |
| APC14 | Tetradecyl- phospho- choline | 379.5 | 14 | Zwitterionic | 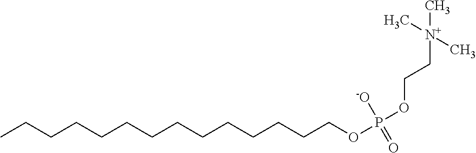 |
| APC16 | Hexadecyl- phospho- choline | 407.5 | 16 | Zwitterionic | 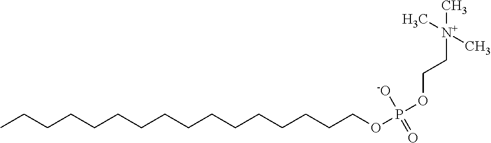 |
| APC18 | Octadecyl- phospho- choline | 435.5 | 18 | Zwitterionic | 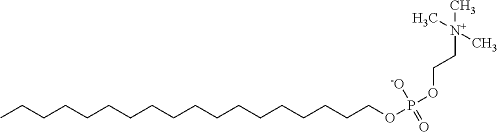 |
| APC11UPC | 10- Undecylenyl- 1- phospho- choline | 335.4 | 11 | Zwitterionic | 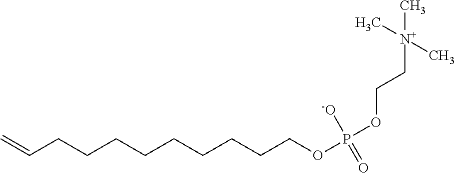 |
| APC11PC | 2,8- Dimethyl-5- Nonyl- phospho- choline | 335.4 | 5 + 5 | Zwitterionic | 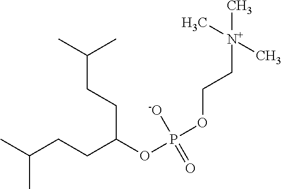 |
| PO | NA | NA | 12 | Anionic | 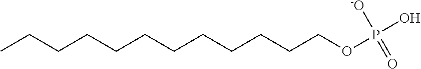 |
| QAC12 | Dodecy- ltrimethyl ammonium bromide | 308.40 | 12 | Cationic | 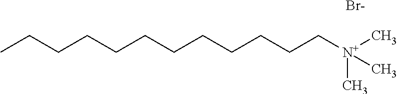 [SMILES: [Br-] ; CCCCCCCCCCCC[N+](C)(C)C] |
| QAC14 | tetradecyl- trimethyl ammonium bromide | 336.40 | 14 | | 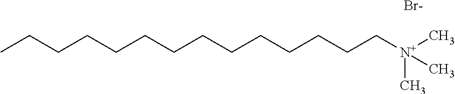 [SMILES: [Br-] ; CCCCCCCCCCCCCC[N+](C)(C)C] |
| QAC16 | hexadecyl- trimethyl ammonium bromide | 264.50 | 16 | | 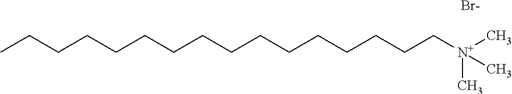 [SMILES: [Br-] ; CCCCCCCCCCCCCCCC[N+](C)(C)C] |
| QAC18 | octadecyl- trimethyl ammonium bromide | 392.50 | 18 | | 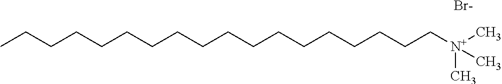 [SMILES: [Br-] ; CCCCCCCCCCCCCCCCCC[N+](C)(C)C] |
| DAB | Didodecyl- dimethyl ammonium bromide | 462.6 | 12 + 12 | Cationic | 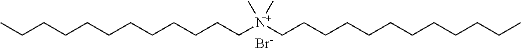 [SMILES: CCCCCCCCCCCC[N+](C)(C)CCCCCCCCCCCC ; [Br-]] |

### Cytotoxicity Assay

The compounds used in this study all contained alkyl-carbon chains: three APCs [dodecyl-PC (APC12), tetradecyl-PC. (APC14) and hexadecyl-PC (APC16)] (Anatrace) and four QACs [docdecyl-TMAB (QAC12), tetradecyl-TMAB (QAC14), hexadecyl-TMAB (QAC16) and octadecyl-TMAB (QAC18)](Sigma). All were prepared to a stock concentration of 1 mg/ml and diluted as required by the experimental protocol in the cytotoxicity assays. A. castellanii trophozoites and cysts (at 10⁵ cells/ml) with or without APCs or QACs (concentrations ranging from 37.35 μg/ml doubly diluted to 0.04 μg/ml to a final volume of 100 μl) in a 96-well plate were incubated at 25° C. for a duration determined by the experimental protocol.

For the combined assay, similar concentrations of QAC18 and APC16 were prepared and 18.75 μg/ml and 37.5 μg/ml of QAC12 added (to a final volume of 100 μl) were added to 10⁵ cells/mi in a 96-well plate and later incubated at 25° C. for a duration determined by the experimental protocol. Resazurin (7-Hydroxy-31-phenoxazin-3-one 10-oxide; 5 mg/ml; McBride J et al (2005) J. Clin. Microbiol.; 43: 629-634) and trypan blue (0.4 μg/ml; Mafra C S P et al (2013) Investig. Ophthalmol. Vis. Si.; 54: 6363-6372) were then added to trophozoites and cysts respectively after being exposed to the compounds and the cell viability estimated microscopically and from changes in absorbance (at 595 nm and 570 nm) respectively.

For the drug kinetic assay, viability was assessed by the linked alamarBlue-reversion assay. Drug-treated cysts were washed with PBS and fresh PG medium was added to allow reversion to trophozoites for 5 days, after which, alamar blue was added and viability estimated as described above.

Efficacy was expressed as a percentage of untreated controls for each drug concentration used and the data used to calculate the IC₅₀. Cell density was estimated using the modified Neubaur hemocytometer and expressed as cells/ml.

### Isolation of Total Genomic DNA

Genomic deoxynucleic acids (gDNA) from 10⁸ A. castellanii trophozoites (treated with and without QAC) were extracted from cells harvested by centrifugation (850×g, 10 min), lysed with UNSET buffer (8M urea, 150 μM NaCl, 2% SDS, 1 μM ethylenediaminetetraacetic acid (EDTA), 100 μM Tris-HCl, pH 7.5) and the DNA extracted with phenol-chloroform (1:1 v/v). The biphasic suspension with DNA enriched on the lower trizol layer was centrifuged (12,000×g for 15 min, 4° C.) for clear separation and transferred to a new microcentrifuge tube. The DNA was precipitated with cold ethanol (100%, v/v) and 0.3M sodium acetate, washed twice with 70% (v/v) ethanol (12,000×g for 10 min, 4° C.), air dried and resuspended in distilled water.

### Morphometric Analysis

A. castellanii trophozoites incubated with and without QAC for a duration determined by experimental protocol were examined microscopically and their sizes measured using the Open Laboratory (Improvision) calibration graticule. The mean body sizes were determined.

### Potassium (K⁺) Determination Assay

Spent medium from A. castellanii trophozoites and cysts was harvested as described above, filtered with the 0.22 μm syringe filter and used for potassium [K⁺] concentration determination using the Atomic Absorption Spectrometry (ASS, absorbance 766.5 nm). A K⁺ standard curve was used to convert absorbance values to concentration (expressed as mg/L).

### Protein and DNA Determination Assay

Protein concentration in cell extract and media of QAC-treated and untreated A. castellanii trophozoites was estimated at 280 nm using the Nanodrop® ND-1000 and expressed as ng/μl protein. DNA concentrations were estimated similarly at 260 nm and expressed as ng/μl.

### OAC-DNA Interaction Assay

The interaction between the gDNA from A. castellanii with QAC12 or QAC18 was investigated at DNA:QAC ratios of 1:0, 1:1, 1:10 and 1:20 for 15 min and the concentration of unreacted DNA in the complex estimated at 260 nm using the Nanodrop® ND-1000 at 260 nm.

### Statistical Analysis

Descriptive statistics of mean and standard deviation values were used to represent data for at least four independent experiments each done in triplicate. To explore differences, between baseline and assay characteristics, T-test statistics were calculated with a statistical threshold of significance set at p<0.01 or p<0.05.

### Results

Long and Short Alkyl-Carbon Chain QACs had Contrasting Activities Against A. castellanii Trophozoites

A. castellanii trophozoites presented contrasting responses with the cationic QACs with alkyl-carbon chain lengths ranging from 12-18 (named QAC12-QAC18). IC₅₀s from QAC14 to QAC18 increased progressively with decreased alkyl-carbon chain lengths (Table 2) with death occurring below their corresponding critical micelle concentration (Table 2).

TABLE 2
IC₅₀s of QACs against protozoans

| | | | | | IC₅₀ | | | |
|---|---|---|---|---|---|---|---|---|
| | | | | | A. castellanii | | A. castellanii | |
| | | No. of | CMC */** | MW | trophozoites | | cysts | |
| Compound | Abbreviation | carbons | (μM) | (g/mol) | μg/ml | μM | μg/ml | μM |
| Dodecyltrimethyl | QAC12 | 12 | ⁺3.5 ± 0.3 | 308.40 | >37.50 | >121.60 | >37.50 | >121.60 |
| ammonium bromide | | | | | | | | |
| tetradecyltrimethyl | QAC14 | 14 | 2.9 ± 0.1 | 336.40 | 9.07 ± 0.14 | 0.27 ± 0.12 | 22.13 ± 1.20 | 0.065 ± 1.04 |
| ammonium bromide | | | | | | | | |
| hexadecyltrimethyl | QAC16 | 16 | 0.26 ± 0.2 | 264.50 | 4.90 ± 0.34 | 0.19 ± 0.03 | 19.17 ± 0.56 | 0.072 ± 0.64 |
| ammonium bromide | | | | | | | | |
| octadecyltrimethyl | QAC18 | 18 | N/A | 392.50 | 1.06 ± 0.03 | 0.03 ± 0.04 | 15.00 ± 0.34 | 0.038 ± 0.24 |
| ammonium bromide | | | | | | | | |

The CMC of QACs collated from *Jalali-Heravi & Konouz, 2003; **Quan et al., 2007 (Lukac et al., 2013). Data is mean ± SD; n = 4 independent experiments performed in triplicates. Student's t-test showed significant difference between all treatments for each parasite life cycle stage at p < 0.01.

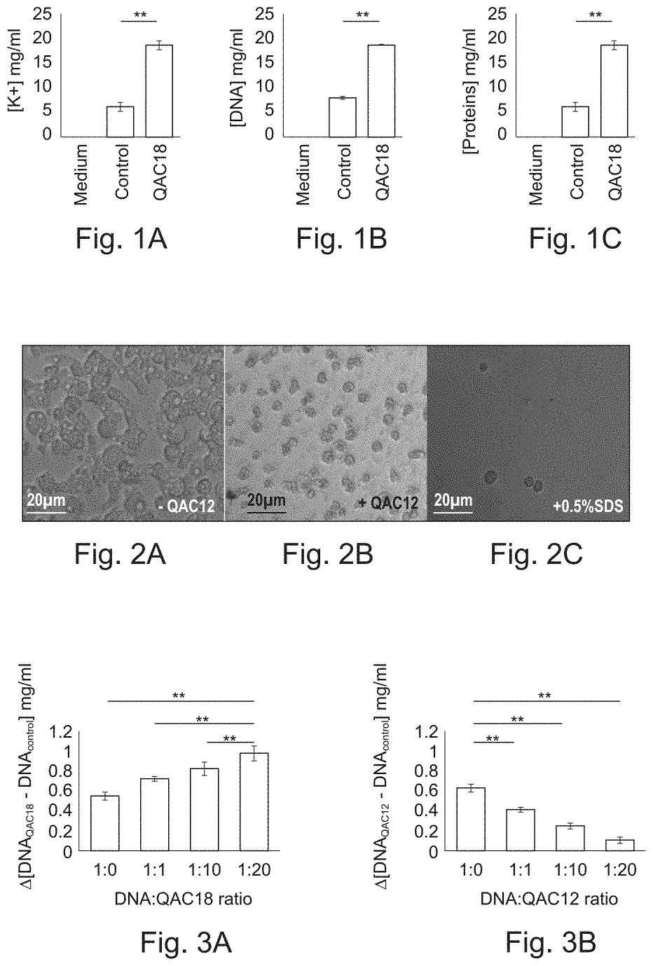

The micelles formed, perhaps on the cell surface, and not in solution (Yaseen M et al (2005) J Colloid Interface Sci; 288(2): 361-70; Isomaa B et al (1979) Acta Pharmacol. Toxicol. (Copenh.); 44(3): 208-15), produce leakage of cytosolic [K⁺], [DNA] and [proteins] from A. castellanii trophozoites into the external milieu, elevated 3-fold (FIG. 1A), 2-fold (FIG. 1B) and 2-fold (FIG. 1C) respectively in treated with QAC18 (at 37.5 μg/ml). Concurrently, the QAC18-treated trophozoites were 4-fold smaller than their untreated counterpart being 7.87±0.93 μm and 29.35±0.23 μm respectively (FIGS. 2A and 2B respectively). Shrunk trophozoites were calcofluor white negative (data not shown) and sensitive to disintegration by 0.5% SDS (92%: FIG. 2C), suggesting that encystation was not occurring. QAC18 wash-out from treated cells and replacement with PG medium was not viable and did not oxidise rezusarin, suggesting that QAC18 and QAC16 were amoebotoxic from 1 h onwards at 37.5 μg/ml respectively: QAC16 and QAC14 was after 24 h at 18.75 μg/ml and only QAC18 was after 24 h at 9.375 μg/ml (Table 2). At all other concentrations, QACs were amoebostatic for up to 96 h (Table 3).

TABLE 3
Baek transformation of QAC-treated cysts to
trophozoites after drag wash-out

| | Drug | Trophozoites | | | | | Cysts | | |
|---|---|---|---|---|---|---|---|---|---|
| | concentration | Hours | | | | | | | |
| Drugs | (μg/ml) | 1 | 24 | 48 | 72 | 96 | 24 | 48 | 72 |
| C14 | 37.50 | + | − | − | − | − | + | + | + |
| | 18.75 | + | + | + | + | + | + | + | + |
| | 9.375 | + | + | + | + | + | + | + | + |
| C16 | 37.50 | − | − | − | − | − | + | − | − |
| | 18.75 | + | − | − | − | − | + | + | + |
| | 9.375 | + | + | + | + | + | + | + | + |
| C18 | 37.50 | − | − | − | − | − | − | − | − |
| | 18.75 | + | − | − | − | − | + | + | + |
| | 9.375 | + | − | − | − | − | + | + | + |
| Control | 0.0000 | + | + | + | + | + | + | + | + |

“+” indicates cells reverting back to trophozoites after washout; and capable of oxidizing reduced alamarBlue from blue to pink after 8 days
“−” indicates cell that did not revert to trophozoites after wash-out and unable to oxidise reduced alamarBlue solution

Examination of the genomic DNA (gDNA) of QAC18-treated A. castellanii trophozoites (18.75 μg/ml) after 96 h using DNA-agarose gel electrophoresis showed that a fragmented DNA profile that depicts death via apoptosis, peculiar of shrunk cells, was not observed (data not shown). However, gDNA of A. castellanii incubated with QAC18 produced a dose-dependent compaction judged by a progressively increased absorbance at 260 nm (FIG. 3A). In contrast, the interaction of QAC12 with A. castellanii gDNA produced a dose-dependent decreased absorbance at 260 nm (FIG. 3B). In control experiments, neither QAC12 nor QAC18 showed absorption at the concentrations used in the interaction assays. Interestingly, a cationic molecule with two 12 alkyl carbons, didodecyldimethyl ammonium bromide (DAB), was inactive while the anionic molecule with similar numbers of alkyl carbon chains, PO (see Table 1), had some residual activity (IC₅₀ of 1100.5±10.5 μg/ml; Table 4).

TABLE 4
IC₅₀s of APC against Acanthamoeba spp

| Compound No | Compound | IC₅₀ (μg/ml) |
|---|---|---|
| 1 | APC8* | 46.4 ± 2.3 |
| 2 | APC10* | 58.0 ± 0.9 |
| 3* | APC12 | 22.9 ± 0.5 |
| 4* | APC14 | 8.9 ± 0.2 |
| 5* | APC16 | 7.9 ± 0.3 |
| 6 | APC18 | 3.0 ± 0.3 |
| 7 | APC6PC | 28.0 ± 1.2 |
| 8 | APC11UPC | 3.1 ± 0.4 |
| 9 | APC11PC | >37.5 |
| Anionic | P0 | 1100.0 ± 10.5 |
| Cationic | DAB | 27.0 ± 2.2 |

*presented in Table 5

Long APCs were Most Toxic Against A. castellanii Trophozoites

The efficacy of the zwitterionic APCs with different alkyl-carbon chain lengths (12-16 carbons) tested against A. castellanii was dependent on tail length, with APC12 and APC16 being the least and most potent with IC₅₀s of 22.90 μg/ml and 7.90 μg/ml respectively (Table 5). APC16-treated cells were 4-fold reduced in size, lacked calcoflour white staining (data not shown), suggesting that encystation was not induced. APC16 wash-out of treated cells, and incubation in PG medium, were viable, able to oxidise resurzarin in cells treated with the drug at between 12.5 μg/ml to 100 μg/ml after 96 h, which points to the compound being amoebostatic (data not shown).

APC Analogue with an Unsaturated Alkyl Carbon

Alterations to the basic APC12 backbone, to introduce a double bond between the first and second carbon atom on the backbone (APC11UPC; IC₅₀ 3.1±0.4 μg/ml) increased activity 3-fold relative to its saturated counterpart, APC12; but this activity was comparable to the longer alkyl carbon chain molecule, APC18 (Table 4).

APC Analogue with a Two Alkyl Carbon Tails

Doubling the number of alkyl carbon tails from one to two, both with a total of 12 carbon atoms (APC11PC) as the parent molecule, APC12 annulled the biological activity under the condition used (Table 4).

APC Analogue with a Longer Linker and Greater Charge Separation

The linker that separates the zwittionic charge was increased from 2 carbon atoms, which are 33A apart, to 6 carbon atoms in the APC12 backbone to produce a molecule with a flexible head named C12P6C. Potency of this molecule was comparable to its parent molecule, APC12 (IC₅₀s 0.28±1.2 μg/ml to 22.9±0.5 μg/ml (Table 4).

PGPubs, use tables from original specification of 2/14/22 (these tables are not cut off).

TABLE 5
IC₅₀s of APCs against Acanthamoeba trophozoites

| | | | | IC₅₀ (μM) | | |
|---|---|---|---|---|---|---|
| | | | | Acanthamoeba | Acanthamoeba | |
| | | No. of | MW | trophozoites | trophozoites | CMC value |
| Compounds | Abbreviation | carbons | g/mol | μg/ml | μM | (μM) |
| Dodecylphosphocholine | APC12 | 12 | 351.50 | 22.90 ± 0.45 | 65.15 ± 0.34 | 1000.00* |
| Tetradecylphosphocholine | APC14 | 14 | 379.50 | 8.90 ± 0.16 | 23.45 ± 0.24 | 120.00* |
| Hexadecylphosphocholine | APC16 | 16 | 407.50 | 7.90 ± 0.26 | 19.39 ± 0.34 | 13.00* |

Activity of APCs against Acanthamoeba trophozoites incubated with different concentrations of APCs for 72 h and 96 h respectively at 26° C. and toxicity determined using alamar blue (Joslin CE et al (2007) Am J Ophthalmol; 144(2): 169-80). The CMCs for APCs were collated from * Yaseen et al. (2005) Biophysical Chemistry; 117: 263-273. Data is mean ± SD; n = 4 independent experiments performed in triplicates. Student's t-test showed significant difference between all treatments for each parasite at p < 0.01.

### QAC12 Delayed Encystation and Enhanced Sensitivity of Trophozoites to APC16 but not QAC18

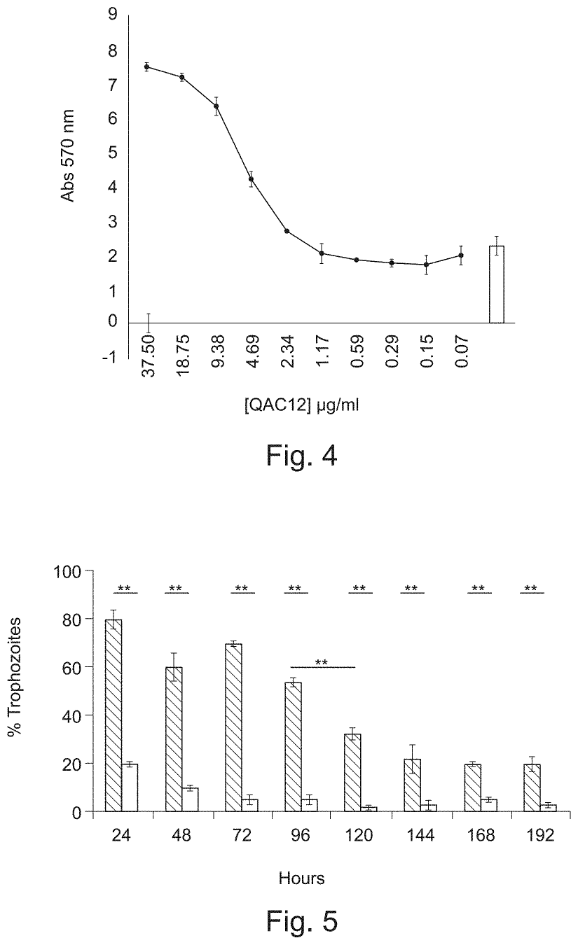

QAC12 was inactive against A. castellanii trophozoites at up to 37.5 μg/ml but, surprisingly, it produced a dose-dependent increased biomass which suggested that it was an energy substrate (FIG. 4) and could provide a favourable condition for trophozoites and delay or stop encystation. The incubation of trophozoites in encystment medium, supplemented with 37.5 μg/ml of QAC12 delayed encystation by 96 h (FIG. 5).

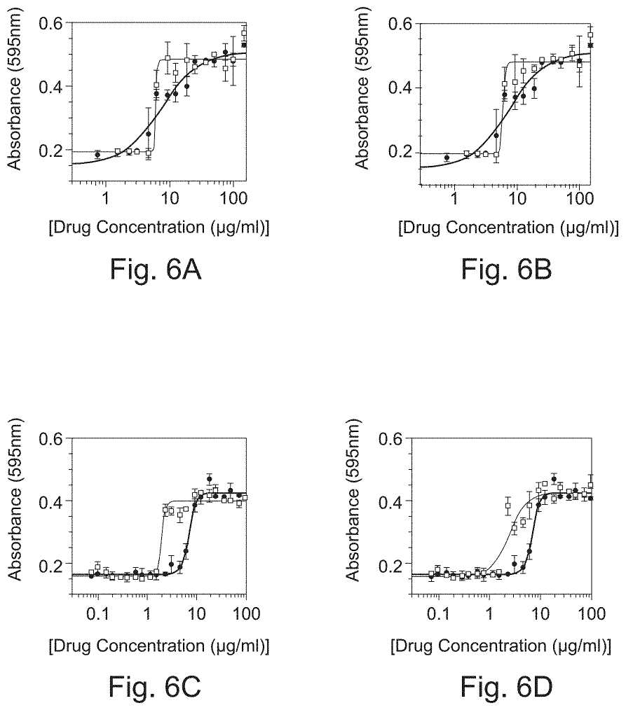

Because QAC12 kept A. castellanii as active replicative trophozoites, it was postulated that, at this state, the trophozoites would be susceptible to the active QACs and APCs. The combination of QAC12 at fixed concentrations of 37.5 μg/ml or 18.75 μg/ml with QAC18 from 100 μg/ml doubly diluted to 0.06 μg/ml produced overlapping dose response curves with their no-drug controls. The estimated IC₅₀s were 6.9 μg/ml and 6.7 μg/ml relative to 6.1 μg/ml respectively (FIGS. 6A and 6B).

In contrast, QAC12 at the same concentrations combined with APC16 (from 100 μg/ml to 0.06 μg/ml) produced shifts in the dose response curves of the QAC12-APC16 treated cells from the APC16 cells (FIGS. 6C and 6D). The estimated IC₅₀s for APC16 with QAC12 at 37.5 μg/ml and 18.75 μg/ml were 2.5 μg/ml and 3.4 μg/ml, statistically significantly different from the APC16 only control experiments (IC₅₀-7.6 μg/ml; p<0.05, FIGS. 6C and 6D). Interestingly, drug wash-out of APC16 treated cells with and without QAC12 showed that APC16 was cytotoxic at 3.12 μg/ml, 4.68 μg/ml, 6.25 μg/ml and 9.36 μg/ml when combined with QAC12 and not alone, judged by their inability to oxidise rezusarin, suggesting that the latter increased the sensitivity of trophozoites to APC16.

QAC12 Interactions were Synergistic for Shorter Alkyl Carbon Chain APC and not their Longer Counterpart

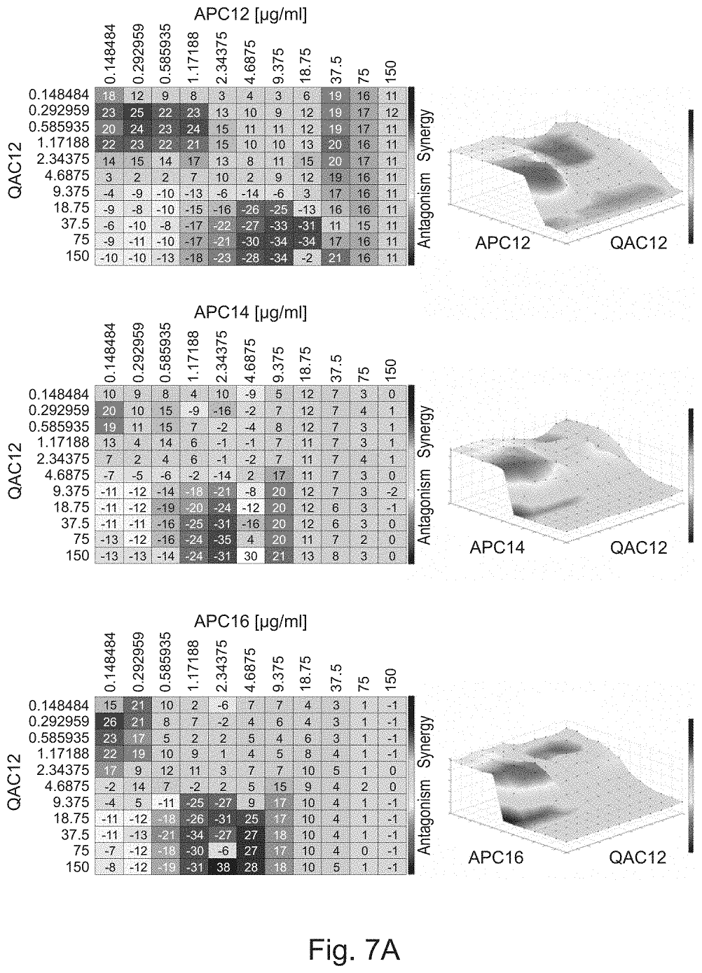

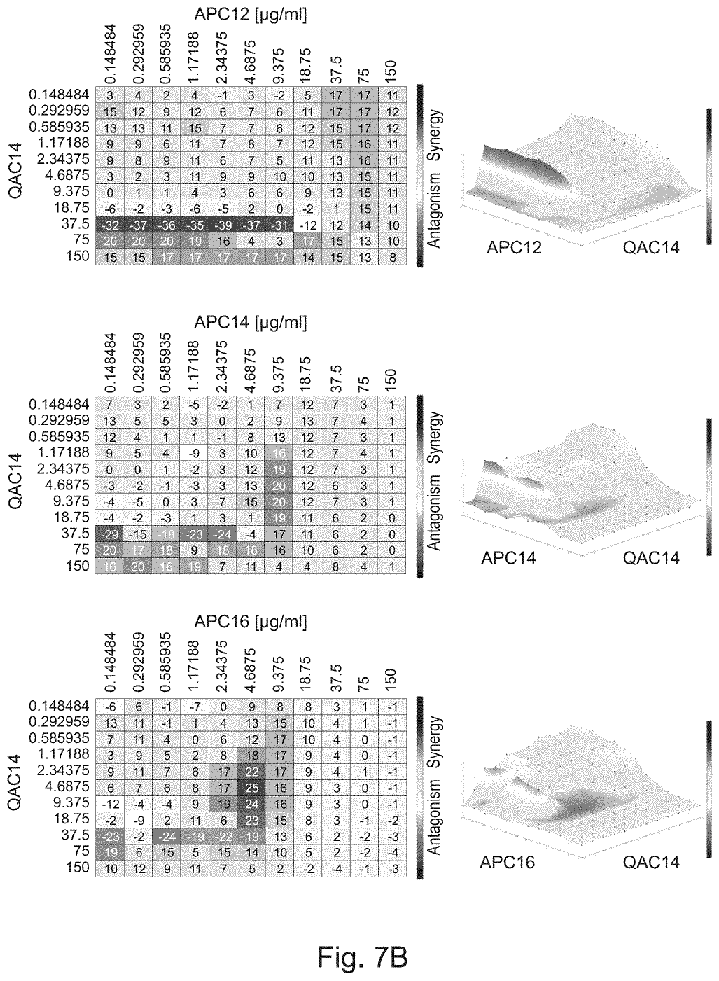

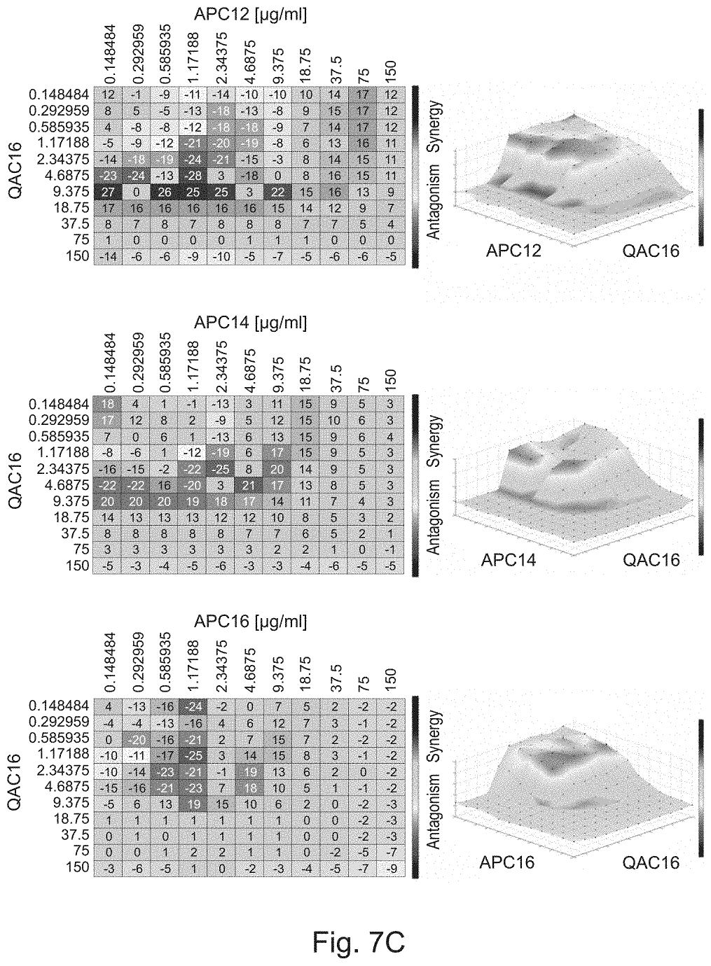

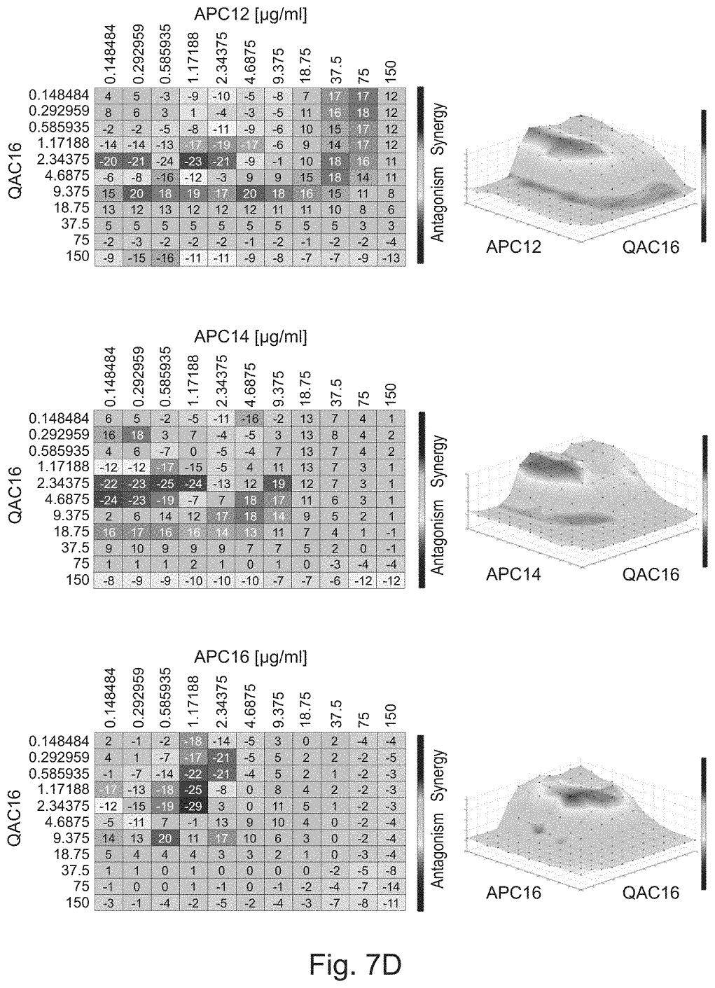

The change in potency between QAC and APC prompted us to used the checkerboard dilution method to identify synergistic interactions (FIG. 7A-D). Three interaction profiles, antagonistic (orange to red), additive (yellow to green) and synergistic (blue) were observed, with optimal synergistic interactions deemed to be significant occurring between 20-38%; (FIG. 7). Significant synergistic ‘well defined’ interactions were more predominant with QAC12-APCs combinations at low to moderate QACs mixed with low to high APCs respectively (FIG. 7A). Significant synergistic interactions were less frequent and ‘noisy’ for QAC14 and QAC16 combined with APCs (FIGS. 7B and 7C). No significant synergy was observed between QAC18 combined with APCs (FIG. 7D).

QACs have Cysticidal Activities Against A. castellanii Cysts

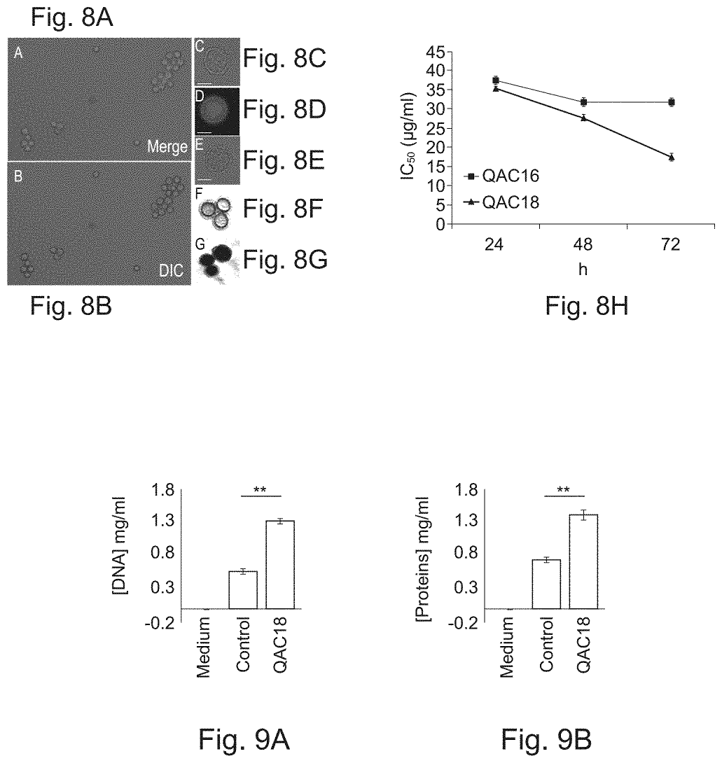

Lastly, we observed that QAC14-QAC18 and not QAC12 had activity against A. castellanii matured cysts, that were calcofluor white positive and SDS-resistant (0.5%, w/v; FIGS. 5A-G), and gave estimated IC₅₀s of 15.00 t 0.06 μg/ml, 19.00±0.03 μg/ml, 22.00±0.04 μg/ml for QAC14, QAC16 and QAC18 after 96 h respectively using the trypan blue viability assay (FIG. 8H, Table 1). The cysticidal activity of QAC16 to QAC18 strongly correlated with (a) the alkyl-carbon chain lengths (Table 2) and the duration of drug contact (FIG. 8H). Like trophozoites, death was via leakage judged by 40% and 50% increase in DNA and proteins in the external milieu after 1 h (FIGS. 9A and 9B). To investigate if the cyst were viable, a complementary viability assay that involved, drug washout, incubation in PG medium to allow reversion of viable cyst to trophozoite for 7 days, and estimation of viability using the oxidation of resazurin after 6 h. No resurgence was observed in cysts incubated with QAC18 and QAC16 at 37.5 μg/ml after 24 h and 48 h respectively and for QAC18 at 18.75 μg/ml it was after 48 h (Table 2).

### Discussion

In this study, the toxicity of APC12, APC14, APC16, APC18, QAC14, QAC16 and QAC18 has been observed against Acanthamoeba trophozoites, with the QACs being more effective than their corresponding APC counterpart, perhaps because they formed larger micelles (QACs, 8.8±0.8 nm; APCs, 5.8±1.0 nm). Interestingly, the activities from both compounds against trophozoites strongly correlated with their alkyl-carbon chain lengths, a key determinant of micellar size. However, the duration of contact between the compound and the protist was most important, with longer alkyl-chain molecules requiring shorter contact times (<24 h) than their shorter counterparts to exert their cytotoxicity. This probably relates to the nature of their interaction, with the abundant complementary alkyl carbon chains in the protist's plasma membrane. Indeed, the abundant fatty acid chains in A. castellanii plasma membranes are 20-30 carbon atoms long (Palusinska-Szysz M et al (2014) PLoS One; 9(7): e101243), presumably enough to produce rapid solubilisation and death.

Increased K⁺, DNA and proteins into the external milieu of trophozoites confirmed that death was produced in part via leakage (FIG. 1), at QAC concentrations below CMCs (Table 1). While it is possible that the cationic QACs and not their zwitterionic counterpart can induce rapid reversal of the net negative charge of the protist plasma membrane to positive with detrimental effect, the DNA compacting caused by QAC18 and not QAC12 suggested that QAC had an intracellular target to effect death in A. castellanii. Unexpectedly, death was not due to apoptosis.

A further unexpected observed was that QAC12 was an energy substrate for A. castellanii trophozoites that increased trophozoites biomass and delayed encystation by 96 h in encystation media. The use of QAC12 for energy prompted its use to maintain the protist as trophozoites, long enough for a second drug to exert its cytotoxic effect in a rational combination therapy. The combination of QAC12 with APC16 increased the sensitivity of trophozoites to the latter. It is possible that the increased uptake of QAC12 for energy could have inadvertently increased APC16 uptake. Also, it is possible that the mixed surfactants, each with a different overall charge, have lowered the surface interfacial tension of the molecule and CMCs, thus increasing the molar solubilisation ratio of the compounds and forming mixed or larger micelles which are lethal.

Another key finding in this study was that QACs were toxic to A. castellanii cysts, also producing death via leaking as a result of conformational changes and weakening of the inter-fibre bonds of the cellulose cell wall. The evidence for this was indirect and based on a time-dependent loss of calcofluor white staining. Indeed, the loss of the natural auto-fluorescent of carboxymethyl cellulose after QAC activity has been linked with conformational changes in cellulose. This correlates with the use of QACs in altering the structural conformation of wood cellulose in paper manufacturing (U.S. Pat. No. 4,144,122).

Finally, it has been shown herein that mixed surfactants, particularly QAC12 mixed with APCs, are efficacious against Acanthamoeba castellanii, enough for their inclusion in contact lens solutions as a preventative management technique of contact lens infection (Hay et al supra; Llull D et al (2007) Antimicrob. Agents Chemother.; 51 (5): 1844-1848; Siddiqui R et al (2014) Pathog. Glob. Health; 108(1): 49-52) and to prevent compliant users from AK. It is suggested that a two stage disinfectant protocol provides an effective preventative management protocol of contact lens care, comparable to existing contact lens solutions, to reduce AK incidence amongst compliant contact lens users.

In summary, cytotoxicity assays and a variety of biophysical approaches have been used to show that alkylphosphocholines (APCs) and quaternary ammonium compounds (QACs) have good efficacy against A. castellanii cysts and trophozoites. Such efficacy was dictated by the length of the alkyl carbon chain lengths, with death occurring in part via leakage and DNA compacting. The QACs were more effective than APCs against trophozoites and were also cytotoxic to cysts. In contrast, QAC12 was an energy substrate that increased A. casiellanii trophozoites biomass, delayed trophozoite-cyst conversion by 96 h and, in combination with APC16 and not QAC18, was synergistic against trophozoites. The results present an effective management strategy for protecting contact lenses from A. casiellanii cysts and trophozoites, and reduce transmission and AK incidence.

The invention may further comprise a concentrate for use in forming a contact lens solution.

Further aspects and/or embodiments of the invention are described in the following clauses:

Clause 1. A pharmaceutical composition comprising at least one anti-Acanthamoeba agent and a physiologically or pharmaceutically acceptable carrier or excipient, for use in the treatment of Acanthamoeba infection, wherein the anti-Acanthamoeba agent has a structure according to formula (I)

R¹—[Lₘ]-N⁺(R²)(R³)(R⁴) (I)

wherein

R¹ is an optionally substituted saturated or unsaturated C₁₀-C₂₄ chain;

L is a linker moiety, wherein m=0 or 1; and

R², R³ and R⁴ are independently C₁-C₄ alkyl substituents (preferably n-alkyl), aryl substituents, or, together with an alkylene moiety of an optional linker L, forms a heterocyclic moiety, whilst optionally one of R², R³ and R⁴ may be a straight-chain, unsubstituted aliphatic hydrocarbon chain of up to 22 having a definition corresponding to that of R¹.

Clause 2. The pharmaceutical composition according to Clause 1, wherein the composition is an acanthamoebocide.
Clause 3. The pharmaceutical composition according to Clause 2, wherein the anti-Acanthamoeba agent kills trophozoite and cyst forms of Acanthamoeba.
Clause 4. The pharmaceutical composition according to any one of clauses 1 to 3, wherein m=0 and there is no linker moiety between the R¹— group and the —N⁺(R²)(R³)(R⁴) head group.
Clause 5. The pharmaceutical composition according to clause 4, wherein the R¹ group is a C₁₄, C₁₆ or C₁₈ straight-chain, unsubstituted aliphatic hydrocarbon chain.
Clause 6. The pharmaceutical composition according to clause 4 or clause 5, wherein the R¹ group has a double bond between the terminal two carbons of the free end of the group.
Clause 7. The pharmaceutical composition according to any one of clauses 1 to 5, wherein the anti-Acanthameoba agent is selected from one or a combination of tetradecyltrimethyl ammonium bromide, hexadecyltrimethyl ammonium bromide and octadecyltrimethyl ammonium bromide.
Clause 8. The pharmaceutical composition according to any one of the preceding clauses, wherein the composition is formulated for intraocular application.
Clause 9. The pharmaceutical composition according to any one of the preceding clauses, wherein the treatment of Acanthamoeba infection prevents or treats Acanthamoeba keratitis.
Clause 10. The pharmaceutical composition according to any one of the preceding clauses, wherein the infectious agent of the Acanthamoeba infection is Acanthamoeba castellani.
Clause 11. The pharmaceutical composition according to any one of the preceding clauses, which further comprises an encystation inhibiting agent.
Clause 12. A contact lens solution comprising at least one anti-Acanthamoeba agent and an acceptable carrier or excipient, wherein the anti-Acanthamoeba agent has a structure according to formula (I)

R¹-[Lₘ]-N⁺(R²)(R³)(R⁴) (I)

wherein

R¹ is an optionally substituted saturated or unsaturated C₁₀-C₂₄ chain;

L is a linker moiety, wherein m=0 or 1; and

R², R³ and R⁴ are independently C₁-C₄ alkyl substituents (preferably n-alkyl), aryl substituents, or, together with an alkylene moiety of an optional linker L, forms a heterocyclic moiety, whilst optionally one of R², R³ and R⁴ may be a straight-chain, unsubstituted aliphatic hydrocarbon chain of up to 22 having a definition corresponding to that of R¹.

Clause 13. The contact lens solution according to clause 12, wherein m=0 and there is no linker moiety between the R¹— group and the —N⁺(R²)(R;)(R⁴) head group.
Clause 14. The contact lens solution according to clause 13, wherein the R¹ group is a C₁₄, C₁₆ or C₁₈ straight-chain, unsubstituted aliphatic hydrocarbon chain.
Clause 15. The contact lens solution according to clause 13 or clause 14, wherein the R¹ group has a double bond between the terminal two carbons of the free end of the group.
Clause 16. The contact lens solution according to any one of clauses 12 to 14, wherein the anti-Acanthameoba agent is selected from one or a combination of tetradecyltrimethyl ammonium bromide, hexadecyltrimethyl ammonium bromide and octadecyltrimethyl ammonium bromide.
Clause 17. The contact lens solution according to any one of Clauses 12 to 16 for use in the treatment of Acanthamoeba infection, or prevention or treatment of Acanthamoeba keratitis.
Clause 18. The contact lens solution according to any one of Clauses 12 to 17, wherein the solution is for use in cleaning contact lenses.
Clause 19. The contact lens solution according to any one of clauses 12 to 18, wherein the infectious agent of the Acanthamoeba infection is Acanthamoeba castellanii.
Clause 20. A pharmaceutical composition comprising:

a) an Acanthamoeba encystation inhibitor;

b) an Acanthamoeba cytotoxic agent; and

c) a physiologically or pharmaceutically acceptable carrier or excipient.

Clause 21. The pharmaceutical composition according to clause 20, wherein the Acanthamoeba encystation inhibitor has a structure define by Formula II:

R⁵-|L_{q}|-N⁺(R₇)(R⁸)(R⁹) (II)

wherein

R¹ is an optionally substituted saturated or unsaturated C₆-C₁₄ aliphatic hydrocarbon chain;

L is a linker moiety, wherein q=0 or 1; and

R⁷, R⁸ and R⁹ are independently C₁-C₆ alkyl substituents, aryl substituents, or, together with an alkylene moiety of an optional linker L, forms a heterocyclic moiety, whilst optionally one of R⁷, R⁸ and R⁹ may be a straight-chain or branched-chain, unsubstituted aliphatic hydrocarbon chain having a definition corresponding to that of R⁶.

Clause 22. The pharmaceutical composition according to clause 21, wherein q=0 and there is no linker moiety between the R⁶— group and the —N⁺(R⁷)(R⁸)(R⁹) head group and wherein the R⁶ group is a straight-chain, saturated, unsubstituted aliphatic hydrocarbon chain having 6 to 12 carbons
Clause 23. The pharmaceutical composition according to clause 22, wherein the R⁶ group has 12 carbon atoms.
Clause 24. The pharmaceutical composition according to clause 23, wherein the Acanthamoeba encystation inhibitor dodecyltrimethyl ammonium bromide.
Clause 25. The pharmaceutical composition according to any one of clauses 20 to 24, wherein the Acanthamoeba cytotoxic agent is an anti-Acanthamoeba agent having a structure according to formula (I)

R¹-[Lₘ]-N⁺(R²)(R³)(R⁴) (I)

wherein

R¹ is an optionally substituted saturated or unsaturated C₁₀-C₂₄ aliphatic hydrocarbon chain;

L is a linker moiety, wherein m=1 and the linker comprises an alkylphosphate group so as to form a —O—P(O₂)⁻O—R⁵— linker between the R¹ group and —N⁺(R²)(R³)(R⁴) group, where R comprises a substituted or unsubstituted, straight-chain or branched —(CH₂)ₚ— group, where p=from 1 to 6; and

R², R³ and R⁴ are independently C₁-C₄ alkyl substituents (preferably n-akyl), aryl substituents, or, together with an alkylene moiety of an optional linker L, forms a heterocyclic moiety.

Clause 26. The pharmaceutical composition according to clause 25, wherein R¹ is an unsubstituted straight-chain aliphatic hydrocarbon of 14, 16 or 18 carbon atoms.
Clause 27. The pharmaceutical composition according to clause 25 or clause 26, wherein the R¹ group a double bond between the terminal two carbon atoms of the free end of the R¹ group.
Clause 28. The pharmaceutical composition according to clause 25 or clause 26, wherein the anti-Acanthamoeba agent is one or a combination of tetradecylphosphocholine, hexadecylphosphocholine or octadecylphosphocholine.
Clause 29. The pharmaceutical composition according to any one of clauses 20 to 28, wherein the composition is an acanthamoebocide.
Clause 30. The pharmaceutical composition according to any one of clauses 20 to 29, wherein the composition is formulated for intraocular application.
Clause 31. The pharmaceutical composition according to any one of clauses 20 to 30, wherein the Acanthamoeba encystation inhibitor and/or the Acanthamoeba cytotoxic agent is specific for Acanthamoeba castellanii.
Clause 32. The pharmaceutical composition according to any one of clauses 20 to 31 for use in the treatment of Acanthamoeba infection.
Clause 33. The pharmaceutical composition according to clause 32, wherein the treatment of Acanthamoeba infection prevents or treats Acanthamoeba keratitis.
Clause 34. A contact lens solution comprising:

a) an Acanthamoeba encystation inhibitor;

b) an Acanthamoeba cytotoxic agent; and

c) an acceptable carrier or excipient.

Clause 35. A contact lens solution according to clause 34, wherein the Acanthamoeba encystation inhibitor and the Acanthamoeba cytotoxic agent are as defined in any one of clauses 21 to 28.
Clause 36. The contact lens solution according to clause 34 or clause 35, wherein the composition is an acanthamoebocide.
Clause 37. The contact lens solution according to any one of 34 to 36, wherein the Acanthamoeba encystation inhibitor and/or the Acanthamoeba cytotoxic agent is specific for Acanthamoeba castellanii
Clause 38. The contact lens solution according to any one of clauses 34 to 37 for use in the treatment of Acanthamoeba infection, or prevention or treatment of Acanthamoeba keratitis.
Clause 39. The contact lens solution according to any one of clauses 34 to 38, wherein the solution is for use in cleaning contact lenses.
Clause 40. The contact lens solution according to clause 38 or clause 39, wherein the infectious agent of the Acanthamoeba infection is Acanthamoeba castellanii.

Each of the embodiments defined by the above clauses may be incorporated, where appropriate in the context, into a pharmaceutical composition or a contact lens solution according to the aspects of the invention set out above.

The invention has been described with reference to preferred embodiments. However, it will be appreciated that variations and modifications can be effected by a person of ordinary skill in the art without departing from the scope of the invention.

1. A pharmaceutical composition comprising:
a) an Acanthamoeba encystation inhibitor;
b) an Acanthamoeba cytotoxic agent; and
c) a physiologically or pharmaceutically acceptable carrier or excipient.

2. The pharmaceutical composition as claimed in claim 1, wherein the Acanthamoeba encystation inhibitor has a structure defined by Formula II:
R⁶—[L_{q}]—N⁺(R⁷)(R⁸)(R⁹) (II)
wherein
R⁶ is an optionally substituted saturated or unsaturated C₆-C₁₄ aliphatic hydrocarbon chain;
L is a linker moiety, wherein q=0 or 1; and
R⁷, R⁸ and R⁹ are independently C₁-C₆ alkyl substituents, aryl substituents, or, together with an alkylene moiety of an optional linker L, forms a heterocyclic moiety, whilst optionally one of R⁷, R⁸ and R⁹ may be a straight-chain or branched-chain, unsubstituted aliphatic hydrocarbon chain having a definition corresponding to that of R⁶.

3. The pharmaceutical composition as claimed in claim 2, wherein q=0 and there is no linker moiety between the R⁶— group and the —N⁺(R⁷)(R⁸)(R⁹) head group and wherein the R⁶ group is a straight-chain, saturated, unsubstituted aliphatic hydrocarbon chain having 6 to 12 carbons

4. The pharmaceutical composition as claimed in claim 3, wherein the R⁶ group has 12 carbon atoms.

5. The pharmaceutical composition as claimed in claim 4, wherein the Acanthamoeba encystation inhibitor comprises dodecyltrimethyl ammonium bromide.

6. The pharmaceutical composition as claimed in claim 1, wherein the Acanthamoeba cytotoxic agent is an anti-Acanthamoeba agent having a structure according to formula (I)
R¹—[Lₘ]—N⁺(R²)(R³)(R⁴) (I)
wherein
R¹ is an optionally substituted saturated or unsaturated C₁₀-C₂₄ aliphatic hydrocarbon chain;
L is a linker moiety, wherein m=1 and the linker comprises an alkylphosphate group so as to form a —O—P(O₂)⁻O—R⁵— linker between the R′ group and —N⁺(R²)(R³)(R⁴) group, where R⁵ comprises a substituted or unsubstituted, straight-chain or branched —(CH₂)ₚ— group, where p=from 1 to 6; and
R², R³ and R⁴ are independently C₁-C₄ alkyl substituents (preferably akyl), aryl substituents, or, together with an alkylene moiety of an optional linker L, forms a heterocyclic moiety.

7. The pharmaceutical composition as claimed in claim 6, wherein R′ is an unsubstituted straight-chain aliphatic hydrocarbon of 14, 16 or 18 carbon atoms.

8. The pharmaceutical composition as claimed in claim 6, wherein the R′ group includes a double bond between the terminal two carbon atoms of the free end of the R¹ group.

9. The pharmaceutical composition as claimed in claim 6, wherein the anti-Acanthamoeba agent is one or a combination of tetradecylphosphocholine, hexadecylphosphocholine or octadecylphosphocholine.

10. The pharmaceutical composition as claimed in claim 1, wherein the composition is an acanthamoebocide.

11. The pharmaceutical composition as claimed in claim 1, wherein the composition is formulated for intraocular application.

12. The pharmaceutical composition as claimed in claim 1, wherein the Acanthamoeba encystation inhibitor and/or the Acanthamoeba cytotoxic agent is specific for Acanthamoeba castellanii.

13. The pharmaceutical composition as claimed in claim 1 for use in the treatment of Acanthamoeba keratitis.

14. A contact lens solution comprising:
a) an Acanthamoeba encystation inhibitor;
b) an Acanthamoeba cytotoxic agent; and
c) an acceptable carrier or excipient.

15. The contact lens solution as claimed in claim 14, wherein the composition is an acanthamoebocide.

16. The contact lens solution as claimed in claim 14, wherein the Acanthamoeha encystation inhibitor and/or the Acanthamoeba cytotoxic agent is specific for Acanthamoeba castellanii.

17. The contact lens solution as claimed in claim 14 for use in the treatment of Acanthamoeba infection, or prevention or treatment of Acanthamoeba keratitis.

18. The contact lens solution as claimed in claim 14, wherein the solution is for use in cleaning contact lenses.

19. The contact lens solution as claimed in claim 17, wherein the infectious agent of the Acanthamoeba infection is Acanthamoeba castellani.

20. A pharmaceutical composition comprising at least one anti-Acanthamoeba agent and a physiologically or pharmaceutically acceptable carrier or excipient, for use in the treatment of Acanthamoeba infection, wherein the anti-Acanthamoeba agent has a structure according to formula (I)
R¹—[Lₘ]—N⁺(R²)(R³)(R⁴) (I)
wherein
R¹ is an optionally substituted saturated or unsaturated C₁₀-C₂₄ chain;
L is a linker moiety, wherein m=0 or 1; and
R², R³ and R⁴ are independently C₁-C₄ alkyl substituents (preferably n-alkyl), aryl substituents, or, together with an alkylene moiety of an optional linker L, forms a heterocyclic moiety, whilst optionally one of R², R³ and R⁴ may be a straight-chain, unsubstituted aliphatic hydrocarbon chain of up to 22 having a definition corresponding to that of R¹.

21. A pharmaceutical composition as claimed in claim 20, wherein the anti-Acanthamoeba agent kills trophozoite and cyst forms of Acanthamoeba.

22. A pharmaceutical composition as claimed in claim 20, wherein m=0 and there is no linker moiety between the R¹— group and the —N⁺(R²)(1.0)(R⁴) head group and the R¹ group is a C₁₄, C₁₆ or C₁₈ straight-chain, unsubstituted aliphatic hydrocarbon chain.

23. A contact lens solution comprising at least one anti-Acanthamoeba agent and an acceptable carrier or excipient, wherein the anti-Acanthamoeba agent has a structure according to formula (I)
R¹—[Lₘ]—N⁺(R²)(R³)(R⁴) (I)
wherein
R¹ is an optionally substituted saturated or unsaturated C₁₀-C₂₄ chain;
L is a linker moiety, wherein m=0 or 1; and
R², R³ and R⁴ are independently C₁-C₄ alkyl substituents (preferably n-alkyl), aryl substituents, or, together with an alkylene moiety of an optional linker L, forms a heterocyclic moiety, whilst optionally one of R², R³ and R⁴ may be a straight-chain, unsubstituted aliphatic hydrocarbon chain of up to 22 having a definition corresponding to that of R¹.

24. The contact lens solution as claimed in claim 23, wherein the infectious agent of the Acanthamoeba infection is Acanthamoeba castellanii.

25. A contact lens solution as claimed in claim 23, wherein the anti-Acanthamoeba agent kills trophozoite and cyst forms of Acanthamoeba.

26. A contact lens solution as claimed in claim 23, wherein m=0 and there is no linker moiety between the R¹— group and the —N⁺(R²)(R³)(R⁴) head group and the R¹ group is a C₁₄, C₁₆ or C₁₈ straight-chain, unsubstituted aliphatic hydrocarbon chain.
